(19) United States
(12) Patent Application Publication
(10) Pub. No.: US 2019/0293634 A1
(43) Pub. Date: Sep. 26, 2019

(54) ISOLATED INTESTINAL MUCOSA AND USES THEREOF

(71) Applicant: Albert LI, Columbia, MD (US)

(72) Inventor: Albert LI, Columbia, MD (US)

(73) Assignee: Albert LI, Columbia, MD (US)

(21) Appl. No.: 16/286,826

(22) Filed: Feb. 27, 2019

Related U.S. Application Data
(60) Provisional application No. 62/636,351, filed on Feb. 28, 2018.

(51) Int. Cl. G01N 33/50; A01N 1/02

(52) U.S. Cl. CPC G01N 33/5088; G01N 33/5014; A01N 1/0221

(57) ABSTRACT

The present disclosure provides an in vitro reagent for evaluating xenobiotic metabolism in a cell culture based assay. The in vitro reagent is an admixture of a cell culture medium and isolated intestinal mucosa comprising villi wherein the intestinal mucosa was eluted from a lumen of the intestine. The isolated mucosa comprises metabolically competent cells. Addition of a xenobiotic test compound to the in vitro reagent allows metabolism of the test compound by the isolated intestinal mucosa comprising villi.

### CROSS-REFERENCE TO RELATED APPLICATIONS

This application claims the benefit of U.S. Provisional Patent Application No. 62/636,351, filed on 28 Feb. 2018, the content of which is incorporated herein by reference in its entirety.

### FIELD OF THE INVENTION

The disclosed invention relates generally to metabolically competent isolated intestinal mucosal comprising villi eluted from intestines, cryopreservation and their use in evaluating biological activity of test substances including drug metabolism activity.

### BACKGROUND OF THE INVENTION

The invention relates to an in vitro experimental system to be used in drug discovery and development for the evaluation of intestinal drug properties (drug metabolism, toxicity, and pharmacology), methods of manufacturing the reagent, methods of cryopreserving the reagent and their methods of use for evaluating biological activity of a test compound.

Being responsible for absorption and metabolism of ingested foods, drugs, and pollutants, the intestines represent an important human organ. Further, intestines represent one of the first organs of defense against pathogenic microbes that are inadvertently ingested with contaminated foods. The intestines also represent a drug target for enteric diseases. An in vitro experimental system of the small intestines would greatly advance our understanding of drug metabolism, toxicology, and pharmacology, and can be an effective tool for drug discovery and development.

The small intestines consist of a tubular organ, with the lumen lined by intestinal mucosa. The intestinal mucosa surface area per length of the intestine is tremendous increased via the organization of the mucosa as intestinal villi intestinal projections into the lumen consists of the mucosal epithelium and blood vessels.

Drug metabolism is an important aspect of drug development. As a drug is administered to the human body, the parent drug is subjected to metabolism by the small intestine before absorption into the portal circulation and subjected to metabolism by the liver. Drug metabolism is critical to the system half-live of the parent drug due to metabolic elimination, toxicity due to the toxicity of the parent drug and metabolites, and efficacy due to the pharmacological effects of the parent drug and metabolites. Due to specific difference in drug metabolism, studies with nonhuman animals do not always provide information useful for the assessment of human outcomes. This is overcome by the use of in vitro human-based experimental systems. Evaluation of hepatic drug metabolism using in vitro human-based systems such as human liver homogenates, microsomes and hepatocytes is now routinely practiced in drug development. Li, A. P. (2004). “In vitro approaches to evaluate ADMET drug properties.” Current topics in medicinal chemistry 4(7): 701-706. However, there are only limited experimental systems of the small intestines. The current experimental systems include intestinal homogenates, cell lines, primary cultures, and cryopreserved enterocytes. Ho, M. D., N. Ring, et al. (2017). “Human Enterocytes as an In Vitro Model for the Evaluation of Intestinal Drug Metabolism: Characterization of Drug-Metabolizing Enzyme Activities of Cryopreserved Human Enterocytes from Twenty-Four Donors.” Drug metabolism and disposition: the biological fate of chemicals 45(6): 686-691. Intestinal microsomes are derived from the homogenates and used for drug metabolism studies. The microsomes have relatively low drug metabolizing enzyme activities due to the degradation of the enzymes during the homogenization process. Cell lines (e.g. Caco-2 cells) are useful for the evaluation of intestinal permeability but are not useful for drug metabolism studies due to the extremely low expression of drug metabolizing enzymes. Hochman, J. H., M. Chiba, et al. (2001). “P-glycoprotein-mediated efflux of indinavir metabolites in Caco-2 cells expressing cytochrome P450 3A4.” The Journal of pharmacology and experimental therapeutics 298(1): 323-330. Cryopreserved enterocytes retain drug metabolizing enzyme activities and therefore represent the most useful experimental system for the evaluation of intestinal drug metabolism (Ho, Ring et al. 2017). However, cryopreserved enterocytes may represent only a selected population of the intestinal mucosa, therefore they may not provide all information required to understand intestinal biology in vivo.

Oral administration is the preferred route of drug delivery due to its convenience and non-invasiveness. Bioavailability of an orally-administered drug is a combination of both enteric and hepatic events. Drug entrance into the enterocytes is determined by drug permeability across the plasma membrane and/or uptake transport, with intracellular enteric drug concentration further defined by enteric drug metabolism and efflux. The fraction of drug that is delivered to the portal vein upon enteric drug absorption is further subjected to absorption into the liver via passive or transporter mediated uptake, with the fraction delivered to the systemic circulation determined by both hepatic drug metabolism and biliary excretion. Furthermore, as the first organ encountering an orally administered drug, the small intestine is the target of adverse effects commonly observed in the liver, namely, drug toxicity and drug-drug interactions.

As the metabolic fate of an orally administered drug is a result of both enteric and hepatic events, accurate definition of both enteric and hepatic drug properties represents an important discipline in drug development. Currently, estimation of human hepatic drug properties is facilitated by the well-established in vitro hepatic experimental systems including human liver microsomes, cDNA-expressed CYP isoforms, and human hepatocytes. Of these in vitro hepatic systems, hepatocytes are considered the “gold standard” due to the complete drug metabolizing enzyme pathways and cofactors. Successful cryopreservation and culturing of human hepatocytes (Li 2007) has allowed the development of numerous effective approaches to evaluate key hepatic drug properties including transporter mediated drug uptake (Shitara, Li et al. 2003; Badolo, Rasmussen et al. 2010), intrinsic hepatic clearance (Di, Trapa et al. 2012; Menochet, Kenworthy et al. 2012; Baudoin, Prot et al. 2013; Peng, Doshi et al. 2016), metabolite profiling (Bursztyka, Perdu et al. 2008), metabolic enzyme pathway identification (Yang, Atkinson et al. 2016), inhibitory and inductive drug-drug interactions (Doshi and Li 2011; Mao, Mohutsky et al. 2012), transporter-mediated drug efflux (Kanda, Takahashi et al. 2018), and hepatotoxicity (Li 2014; Li 2015; Zhang, He et al. 2016). Cryopreserved human hepatocytes used in conjunction with cell-free systems such as human liver microsomes, cDNA-derived P450 isoforms and transporter membrane vesicles have allowed an accurate assessment of hepatic drug properties. PBPK approaches in combination with various databases and software have been applied successfully for the translation of in vitro observations to clinical events (Shaffer, Scialis et al. 2012; Marsousi, Desmeules et al. 2018).

Development of enteric experimental approaches like that for the definition of hepatic drug properties would further enhance our ability to develop drugs with optimal clinical properties. Recently we have demonstrated successful cryopreservation of partially purified human enterocytes isolated via collagenous digestion of the small intestine (Ho, Ring et al. 2017). The cryopreserved human enterocytes exhibit various drug metabolizing enzyme activities including various P450 and non-P450 pathways and have been used in the evaluation of intestinal clearance drug-drug interaction potential of orally administered drugs with promising results (Yan, Wong et al. 2017).

The invention disclosed herein addresses those deficiencies of enterocytes disclosed above and provides an improvement for evaluating xenobiotic metabolism using the present in vitro reagent; the invention is derived from the intestinal mucosa, thereby containing the key cell types responsible of intestinal drug uptake and metabolism, as well as being the major target for drug-induced enterotoxicity and is the site of action of drugs for enteric diseases. The invention comprises the use of the isolated intestinal mucosa comprising villi (in vitro reagent), which can be used as freshly isolated, or after thawing from cryopreservation, as a reagent for the evaluation of enteric drug uptake, metabolism, toxicity, and pharmacology. The present in vitro reagent represents a physiologically relevant model of the small intestinal mucosa for investigations of enteric drug properties, including drug metabolism, drug-drug interactions, entertoxicity, and enteric pharmacology.

### SUMMARY OF THE INVENTION

Herein are provided an in vitro reagent, manufacturing of the in vitro reagent and methods for evaluating xenobiotic test compound metabolism using the in vitro reagent.

In embodiments provided herein is an in vitro reagent for evaluating biological activity of a test substance, wherein the reagent is a cryopreserved mixture comprising a cell culture medium comprising a cryoprotectant; and metabolically competent isolated intestinal mucosa comprising villi wherein the intestinal mucosa was eluted from an intestine. In embodiments, the intestinal mucosa is eluted from a mammalian or human intestine. In certain embodiments, the metabolically competent isolated intestinal mucosa comprising villi are pooled from more than one donor.

In embodiments, the metabolically competent isolated intestinal mucosa comprises enterocytes. In certain embodiments, the in vitro reagent is thawed prior to use for evaluating biological activity of a test substance. In certain other embodiments, the in vitro reagent is provided in a kit and further comprises instructions for evaluating biological activity of a test substance using the reagent.

In embodiments, the in vitro reagent comprises human metabolically competent isolated intestinal mucosa comprising villi. In further embodiments, that in vitro reagent comprises human metabolically competent isolated intestinal mucosa comprising villi from a pool of human donors. In certain embodiments, the pool of human donors is reflective of a heterogenous population.

In certain embodiments, the present in vitro reagent is prepared wherein the metabolically competent isolated intestinal mucosa comprising villi are eluted from intestines. In embodiments, that method comprises the steps of eluting the intestinal mucosal from lumen of the intestines to form isolated intestinal mucosa; suspending the isolated intestinal mucosa in a cell culture medium; adding a cryoprotectant to the cell culture medium to form an in vitro reagent; and, storing the in vitro reagent frozen at a temperature of −10° C. to about −175° C. (in liquid nitrogen).

In embodiments, the method further comprises use of an enzyme in step a) (e.g. collagenase or a protease). In embodiments, the isolated intestinal mucosa comprising villi are eluted from mammal or human intestines. In embodiments, the metabolically competent isolated intestinal mucosa comprises enterocytes. In embodiments, the methods step of a) and b) are repeated for multiple donor intestine and then pooled wherein step c) is performed. Accordingly, the present method of manufacture provides metabolically competent isolated intestinal mucosa comprising villi pooled from more than one donor. In embodiments, the donors are human, wherein the metabolically competent isolated intestinal mucosa comprising villi are human.

In embodiments, the isolated intestinal mucosa comprising villi (freshly isolated or cryopreserved) are used in methods for evaluating biological activity of a test substance. In certain embodiments, that method comprises the steps of providing metabolically competent isolated intestinal mucosa comprising villi wherein the intestinal mucosa was eluted from a lumen of an intestine; culturing the isolated intestinal mucosa in a cell culture vessel incubated at 30-45° C.; introducing the test substance into the cell culture vessel; incubating the test substance for 0.5 h to 10 days at 30-45° C.; and, performing an end point assay of the isolated intestinal mucosa or cell culture medium to determine biological activity of the test substance.

In one embodiment, the metabolically competent isolated intestinal mucosa is freshly isolated. In an alternative embodiment, the metabolically competent isolated intestinal mucosa is cryopreserved and further comprises a thawing step prior to the culturing step.

In embodiments, the biological activity is metabolism, toxicity, genotoxicity, carcinogenicity, drug-drug interactions, receptor binding, receptor inhibition, biochemical function, gene expression, protein expression, or pharmacological activities.

In embodiments, the methods further comprise measuring the inhibition or induction of cytochrome P450. In certain embodiments, the methods further comprise measuring parent test substance disappearance and metabolite formation of the test substance in the cell culture medium.

In certain embodiments the end point assay is a mammalian genotoxicity assay; a mammalian cytotoxicity assay; or a mammalian or pharmacological assay.

In embodiments, the cell culture vessel comprising the in vitro reagent and test substance is incubated at 37° C.

In embodiments, the test substance is a drug or drug candidate; or an environmental pollutant. In certain embodiments, the drug or drug candidate is selected from the group consisting of an organic compound, an inorganic compound, a hormone, a growth factor, a cytokine, a reception, an antibody, an enzyme, a peptide, a NSAID, an aptomer and a vaccine. In certain embodiments, the drug or drug candidate is an orally administered substance. In embodiments, the drug or drug candidate is added at cytotoxic concentrations for identifying and profiling metabolites.

In certain embodiments, complete phase 1 oxidation and phase 2 conjugation of metabolites are evaluated with the in vitro reagent (e.g., metabolically competent isolated intestinal mucosa comprising villi wherein the intestinal mucosa was eluted from a lumen of an intestine).

In embodiments the methods further comprise evaluating metabolic stability, metabolite profiling and identification, enzyme inhibition or metabolic activation of proto-toxicants or pro-mutagens.

In certain embodiments, the isolated intestinal mucosa comprising villi (freshly isolated or cryopreserved) are used in methods for evaluating in vitro inhibition or induction of cytochrome P450 by a test compound that is ingested or administered orally. In certain embodiments, that method comprises the steps of a) providing metabolically competent isolated intestinal mucosa comprising villi wherein the intestinal mucosa was eluted from an intestine; b) culturing the isolated intestinal mucosa in a cell culture vessel incubated at 30-45° C.; c) introducing a test compound into the cell culture vessel that is suspected of being a P450 ligand; d) incubating the test compound for 1 minute to 10 days at 30-45° C.; and, e) performing an end point assay of the isolated intestinal mucosa or cell culture medium to determine P450 activity.

In embodiments, the test compound suspected of being a P450 ligand is a substrate of P450. In other embodiments, the test compound suspected of being a P450 ligand is an inhibitor or inducer of P450.

In certain embodiments, the isolated intestinal mucosa comprising villi (freshly isolated or cryopreserved) are used in methods for evaluating in vitro intestinal toxicity of a xenobiotic test compound that is ingested or administered orally. In certain embodiments, that method comprises the steps of a) providing metabolically competent isolated intestinal mucosa comprising villi wherein the intestinal mucosa was eluted from an intestine; b) culturing the isolated intestinal mucosa in a cell culture vessel incubated at 30-45° C.; c) introducing a xenobiotic test compound into the cell culture vessel; d) incubating the xenobiotic test substance for 1 minute to 10 days at 30-45° C.; and, e) performing an end point assay of the isolated intestinal mucosa or cell culture medium to determine viability.

In certain embodiments, the isolated intestinal mucosa comprising villi (freshly isolated or cryopreserved) are used in methods for evaluating in vitro intestinal pharmacological effects of a xenobiotic test compound that is ingested or administered orally. In certain embodiments, that method comprises the steps of a) providing isolated intestinal mucosa comprising villi wherein the intestinal mucosa was eluted from an intestine; b) culturing the isolated intestinal mucosa in a cell culture vessel incubated at 30-45° C.; c) introducing a xenobiotic test compound into the cell culture vessel; d) incubating the xenobiotic test compound for 1 minute to 10 days at 30-45° C.; and, e) performing an end point assay of the isolated intestinal mucosa or cell culture medium to determine pharmacological effects.

### BRIEF DESCRIPTION OF THE DRAWINGS

FIG. 1 shows a diagram of an intestinal villus of the mucosa.

FIG. 2 shows a photomicrograph of cryopreserved intestinal mucosa with multiple villi (left panel) and a photomicrograph of homogenized intestinal mucosa following collagen digestion (right panel).

FIG. 3 shows a time-dependent increases in metabolite (luciferin) formation quantified by luminescence from metabolism of the CYP3A4 substrate, luciferin IPA, by cryopreserved isolated intestinal mucosa comprising villi demonstrating drug metabolism activity.

FIG. 4 shows rifampin (20 uM) induction of P450 (CYP3A4) activity in cryopreserved human isolated intestinal mucosa comprising villi.

FIG. 5 shows P450 activities of freshly isolated and cryopreserved isolated human intestinal mucosa comprising villi. See Example 4 and 5.

FIG. 6 shows the dose dependent enterotoxicity activity of naproxen in isolated intestinal mucosa comprising villi.

FIG. 7 shows the dose dependent enterotoxicity activity of acetaminophen naproxen in isolated intestinal mucosa comprising villi.

FIG. 8 shows Acetaminophen (APAP) and Naproxen Enterotoxicity in Cryopreserved Human Intestinal Mucosa from Three Donors with IC50 Values (mg/mL).

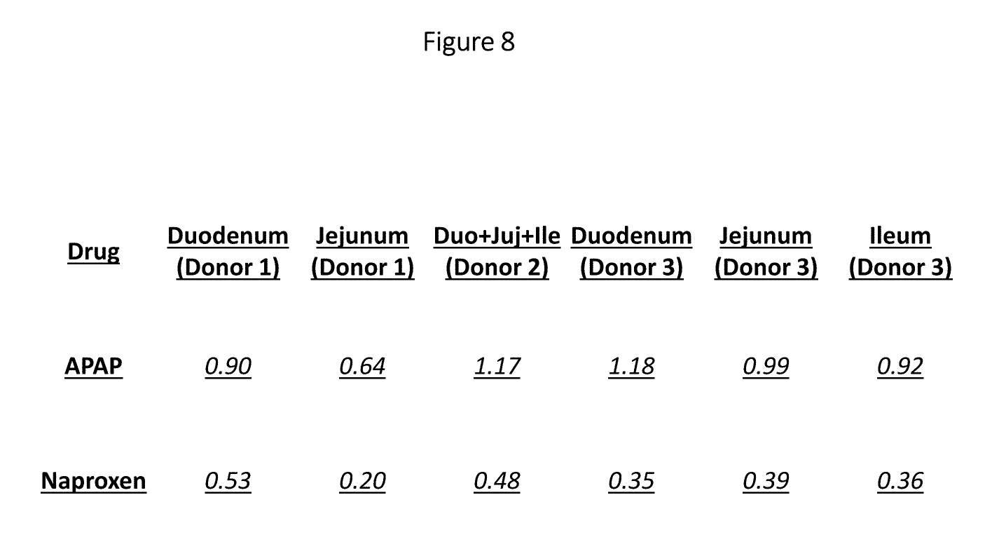

FIG. 9A shows P450 isoform-specific activities of cryopreserved isolated intestinal mucosa comprising villi isolated from the duodenum, jejunum and ileum of Donor 1, Donor 2 and Donor 4, and from the small intestine (from duodenum to ileum) of Donor 3 (BQL=below limits of quantification). See Example 8.

FIG. 9B shows distribution of P450 isoform activities in cryopreserved isolated intestinal mucosa comprising villi using midazolam-1′hydroxylation as CYP3A4 activity. Results are expressed as percent of the arithmetic sum of the specific activities (pmol/min/mg protein) of the P450 isoforms evaluated.

FIG. 9C shows distribution of P450 isoform activities in cryopreserved isolated intestinal mucosa comprising villi using testosterone 6b-hydroxylation (bottom chart) as CYP3A4 activity. Results are expressed as percent of the arithmetic sum of the specific activities (pmol/min/mg protein) of the P450 isoforms evaluated.

FIG. 10A shows Induction of CYP3A4 transcription by rifampin. Error bars represent the standard deviations of results from triplicate treatments

FIG. 10B shows Induction of CYP3A4 transcription by and 1,25(OH)2D3. Error bars represent the standard deviations of results from triplicate treatments

FIG. 10C shows induction of CYP24A1 transcription by 1,25(OH)2D3. Error bars represent the standard deviations of results from triplicate treatments.

FIG. 11 shows non-P450 drug metabolizing enzyme activities of cryopreserved isolated intestinal mucosa comprising villi isolated from the duodenum, jejunum and ileum of Donor 1, Donor 2 and Donor 4, and from the entire small intestine (from duodenum to ileum) of Donor 3 (BQL=below limits of quantification).

FIG. 12 shows Acetaminophen (APAP) enterotoxicity in cryopreserved isolated intestinal mucosa comprising villi isolated from the duodenum, jejunum and ileum of Donor 1.

FIG. 13 shows naproxen cryopreserved isolated intestinal mucosa comprising villi. The results show that naproxen was consistently more toxic than APAP, consistent with human in vivo findings. IC₅₀ ratio was calculated by dividing the IC₅₀ of APAP by that of naproxen.

### DETAILED DESCRIPTION OF THE INVENTION

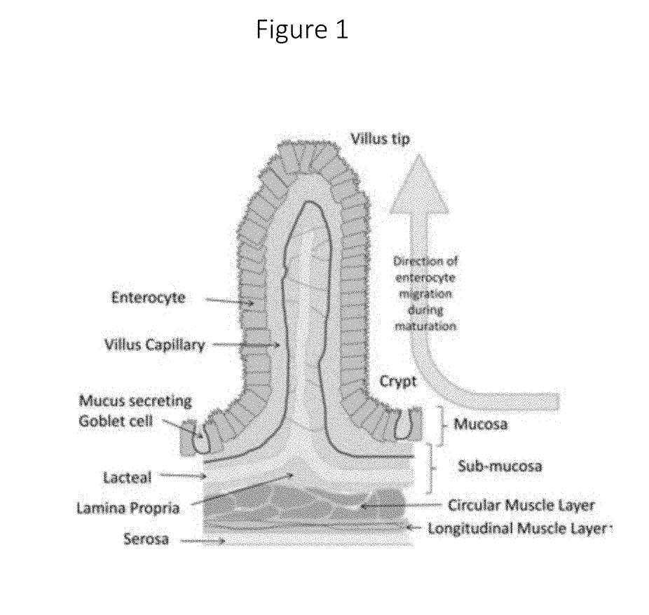

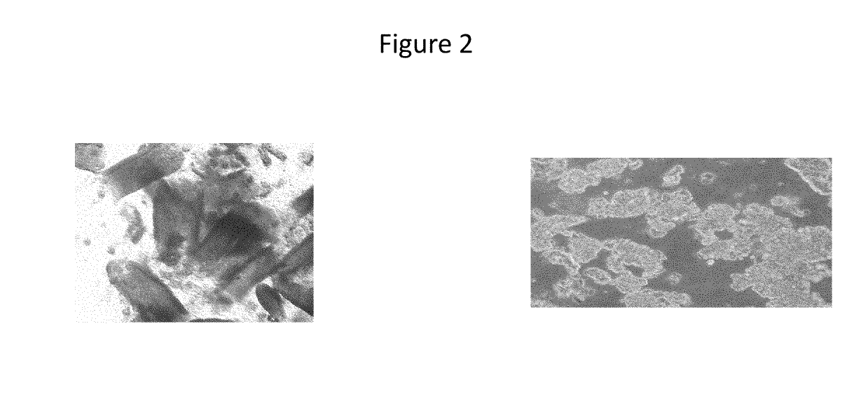

Provided herein are compositions, methods of manufacturing, methods of use and kits for evaluating xenobiotic metabolism; provided herein is an in vitro intestinal experimental system. Intestinal mucosa comprising villi are isolated from human small intestines via elution of the intestinal lumen. See FIGS. 1 and 2. That isolated intestinal mucosa composing villi retain their metabolism activity after cryopreservation or culture for multiple days. See Examples 3-8. In embodiments, the in vitro reagent comprises metabolically competent isolated intestinal mucosa comprising villi. In embodiments, the in vitro reagent (isolated intestinal mucosa comprising villi) can be cryopreserved. Intestinal mucosa recovered after cryopreservation retain drug metabolizing enzyme activities. Successful cryopreservation of the intestinal mucosa allows this experimental system to be readily stored and transported to laboratories other than that involved in the isolation and cryopreservation. The isolated intestinal mucosa contains all cell types that are responsible for interaction with ingested substances (not just enterocytes), thereby representing a physiologically relevant experimental system for the evaluation of intestinal biology. See Example 8. The in vitro reagent comprises metabolically competent cells.

The present in vitro reagent comprising isolated intestinal mucosa comprising villi provides benefits and advantages over use of organelle systems and intact enterocytes. In embodiments, the composition is an in vitro reagent that provides improved means for testing xenobiotic metabolism of orally ingested test substances. In certain embodiments, the in vitro reagent is a cryopreserved mixture comprising a cell culture medium comprising a cryoprotectant; and, an isolated intestinal mucosa comprising villi wherein the intestinal mucosa was eluted from an intestine. In certain embodiments, collagenase is used to elute the intestinal mucosa from the intestine. As used herein, cryopreserved refers to preserving a mixture of cells including villi (i.e. present in vitro reagent) that have been frozen at a temperature of about −10° C. or less, such as about −20° C. to about −175° C. (in liquid nitrogen). In embodiments, the in vitro reagent is frozen in a liquid nitrogen freezer.

In certain embodiments, the in vitro reagent is freshly isolated and does not contain a cryoprotectant. In certain other embodiments, the in vitro reagent is thawed and the cryoprotectant removed by changing the cell culture medium prior to use in methods for evaluating test compounds.

As used in the art, a cryoprotectant or cryopreservative, is a substance that prevents damage to cells and/or organelles during freezing and, includes but is not limited to glycols; such as glycerol, ethylene glycol and propylene glycol; and dimethyl sulfoxide (DMSO). In embodiments, the cryoprotectant comprises glycerol, ethylene glycol, propylene glycol or dimethyl sulfoxide (DMSO).

As used in the art, cell culture medium is a liquid designed to support the culturing of the cells and for incubation for drug metabolism, toxicity, and pharmacology studies. Cell culture medium for maintaining cells of the isolated villi include Kreb's Hensleit Buffer (KHB), William's E medium, Dulbecco's Modified Eagle's Medium (DMEM), Hepatocyte-Enterocyte Incubation Medium (HQM), and Hepatocyte Induction Medium (HIM).

In certain embodiments the isolated intestinal mucosa is human isolated intestinal mucosa comprising villi. In certain embodiments, the intestinal mucosa comprising villi are isolated from mammalian and non-mammalian animals including rodents (e.g., mice and rats), rabbits, guinea pig, hamsters, pigs, cows, horses, dogs, monkeys, fish, reptiles, and amphibians.

The present in vitro reagent comprised of isolated intestinal mucosa comprising villi, represent a complete system with all the drug metabolizing enzymes present in enterocytes and non-enterocytes of the villi. The present in vitro reagent can be stored for a prolonged duration (years) in liquid nitrogen, and for shorter durations (weeks) in a regular laboratory freezer at −10 or −80° C. In embodiments, the in vitro reagent is stored in a liquid nitrogen freezer. In embodiments, the in vitro reagent can be thawed, cultured and used directly, or used within a few days of being isolated.

In embodiments, the present composition is an in vitro experimental system, wherein the system comprises: a) a cell culture medium admixture comprising; i) cell culture medium; and, ii) isolated intestinal mucosa comprising villi wherein the intestinal mucosa was eluted from an intestine. In certain embodiments, the admixture further comprises a cryoprotectant. The cell culture admixture is also referred to herein as the thawed in vitro reagent. In embodiments, the in vitro reagent is added to a cell culture assay comprising a test compound, incubated for a designated time duration, and quantification of the designated biological effects in the target cells or cell culture medium, thereby allowing the evaluation of the biological effects of an orally ingested test substance. Examples of endpoints can be the quantification of the added test article for the evaluation of enteric metabolic stability, quantification of metabolites from the test article for the evaluation of metabolic fates, quantification of mucosa viability for the evaluation of enteric toxicity of the test article, quantification of enteric drug metabolizing enzyme activities (inhibition or induction) for the evaluation of the drug-drug potential of the test article, and quantification of specific biochemical pathways (enzyme activity, protein expression, gene expression) for the evaluation of pharmacological effects of the test article.

In embodiments, the manufacturing of the instant in vitro reagent is carried out by eluting mucosa comprising villi from the lumen of an intestine. Methods for eluting the mucosa include elution of the intestinal mucosa from the lumen of intestines by flowing cold culture medium through the lumen of the intestines for the dissociation and collection of the released mucosa; or by adding an enzyme solution (e.g. collagenase) in the lumen, followed by incubation at 37° C., and elution of the released mucosa with culture medium; or by adding a chelating agent (e.g. EGTA) in the lumen, followed by incubation at 4° C. or 37° C., and elution of the released mucosa with culture medium. (van Corven, E. J., M. D. de Jong, et al. (1986). “Enterocyte isolation procedure specifically effects ATP-dependent Ca2+-transport in small intestinal plasma membranes.” Cell calcium 7(2): 89-99; Nguyen, T. D., J. P. Broyart, et al. (1987). “Laterobasal membranes from intestinal epithelial cells: isolation free of intracellular membrane contaminants.” The Journal of membrane biology 98(3): 197-205; Hitchin, B. W., P. R. Dobson, et al. (1991). “Measurement of intracellular mediators in enterocytes isolated from jejunal biopsy specimens of control and cystic fibrosis patients.” Gut 32(8): 893-899; Lundqvist, C., M. L. Hammarstrom, et al. (1992). “Isolation of functionally active intraepithelial lymphocytes and enterocytes from human small and large intestine.” Journal of immunological methods 152(2): 253-263; Llana, T. and R. G. Bell (1993). “Characterization of an inhibitory factor derived from epithelial cells of the small intestine.” Regional immunology 5(1): 18-27; Keelan, M., E. Wierzbicki, et al. (1995). “Age of rat influences isolation of jejunal enterocytes from along the villus.” Canadian journal of physiology and pharmacology 73(10): 1437-1450). One of skill in the art understands various methods exist for eluting mucosa from the lumen of an intestine, provided the isolated mucosa comprise villi that are metabolically competent. See Example 1. The isolated intestinal mucosa comprising villi can be tested for metabolic activity using, for example, a substrate of P450. See Example 3 and 8.

In embodiments, the in vitro reagent is frozen in liquid nitrogen, comprises a cryoprotectant (e.g., glycerol and/or DMSO and/or serum). In embodiments, the in vitro reagent is manufactured comprising the steps of eluting the intestinal mucosa from lumen of the intestines to form isolated intestinal mucosa; suspending the isolated intestinal mucosa in a cell culture medium; adding a cryoprotectant to the cell culture medium to form an in vitro reagent; and, storing the in vitro reagent frozen at a temperature of −10° C. to about −175° C. (liquid nitrogen).

In embodiments, the in vitro reagent is not prepared for freezing, wherein the in vitro reagent is manufactured comprising the steps of eluting the intestinal mucosa from lumen of the intestines to form isolated intestinal mucosa; and suspending the isolated intestinal mucosa in a cell culture medium. The in vitro reagent may also not comprise a cryoprotectant wherein the in vitro reagent is thawed, following preparation as disclosed above, and the cell culture medium comprising a cryoprotectant exchanged with fresh cell culture medium that does not comprise a cryoprotectant.

The methods of use herein are carried out by addition of a xenobiotic test compound or test article to the in vitro reagent (fresh or previously cryopreserved admixture of a cell culture medium comprising a cryoprotectant and isolated intestinal mucosa comprising villi wherein the intestinal mucosa was eluted from an intestine), and culturing at about a physiological temperature (e.g. about 30° C. to 45° C.), in a cell culture vessel for period of time from about 1 minute to about 10 days. The test compound is introduced into the cell culture vessel comprising the in vitro reagent, incubated, followed by evaluation of the effects of metabolism on the test compound including quantification of the parent compound for the evaluation of metabolic stability; metabolite quantification and identification; evaluation of drug metabolizing enzyme (e.g. P450) activity for evaluation of drug-drug interaction potential, evaluation of mucosal viability for the evaluation of enterotoxicity, and evaluation of mucosal biochemical effects (e.g. receptor binding, gene expression, protein expression, enzyme activity) for the evaluation of pharmacological effects.

In embodiments, the biological activity is metabolism, toxicity, genotoxicity, carcinogenicity, drug-drug interactions, receptor binding, receptor inhibition, biochemical function, gene expression, protein expression, or pharmacological activities. In certain embodiments, the methods comprise measuring the inhibition or induction of cytochrome P450 or measuring parent test substance disappearance and metabolite formation of the test substance in the cell culture medium.

In certain embodiments, provided herein are methods for evaluating in vitro inhibition or induction of cytochrome P450 by a xenobiotic that is ingested or administered orally, comprising: providing isolated intestinal mucosa comprising villi wherein the intestinal mucosa was eluted from an intestine; culturing the isolated intestinal mucosa in a cell culture vessel incubated at 30-45° C.; introducing a xenobiotic test compound into the cell culture vessel that is suspected of being a P450 ligand; incubating the xenobiotic test substance for 1 minute to 10 days at 30-45° C.; and, performing an end point assay of the isolated intestinal mucosa or cell culture medium to determine P450 activity, gene expression, and/or protein expression. In embodiments, the xenobiotic test compound suspected of being a P450 ligand is a substrate of P450. In certain embodiments, the xenobiotic test compound suspected of being a P450 ligand is an inhibitor or inducer of P450.

In certain embodiments provided herein are methods for evaluating in vitro intestinal toxicity of a xenobiotic test compound or substance that is ingested or administered orally, comprising providing isolated intestinal mucosa comprising villi wherein the intestinal mucosa was eluted from an intestine; culturing the isolated intestinal mucosa in a cell culture vessel incubated at 33-40° C.; introducing a xenobiotic test compound into the cell culture vessel; incubating the xenobiotic test substance for 0.5 h to 10 days at 0-45° C.; and, performing an end point assay of the isolated intestinal mucosa or cell culture medium to determine viability.

In certain embodiments provided herein are method for evaluating in vitro intestinal pharmacological effects of a xenobiotic that is ingested or administered orally, comprising: providing isolated intestinal mucosa comprising villi wherein the intestinal mucosa was eluted from an intestine; culturing the isolated intestinal mucosa in a cell culture vessel incubated at 33-40° C.; introducing a xenobiotic test compound into the cell culture vessel; incubating the xenobiotic test substance for 0.5 h to 10 days at 30-45° C.; and, performing an end point assay of the isolated intestinal mucosa or cell culture medium to determine pharmacological effects which may include receptor binding, gene expression, protein expression, and enzyme activity.

The (xenobiotic) test compounds used in the present invention include, but are not limited to drugs, drug candidates, biologicals, food components, herb or plant components, proteins, peptides, oligonucleotides, DNA and RNA. In embodiments, the test compound is a drug, a drug candidate, an industrial chemical, an environmental pollutant, a pesticide, an insecticide, a biological chemical, a vaccine preparation, a cytotoxic chemical, a mutagen, a hormone, an inhibitory compound, a chemotherapeutic agent or a chemical. In certain embodiments, the drug or drug candidate is selected from the group consisting of an organic compound, an inorganic compound, a hormone, a growth factor, a cytokine, a reception, an antibody, an enzyme, a peptide, a NSAID, an aptamer or a vaccine. The test compound can be either naturally-occurring or synthetic and can be organic or inorganic. A person skilled in the art will recognize that the test compound can be added to the in vitro reagent present in the cell culture medium in an appropriate solvent or buffer.

In embodiments, the metabolically competent isolated intestinal mucosa comprising villi comprise enterocytes. Enterocyte metabolism is known to be a major determinant of metabolism-dependent xenobiotic toxicity. P450 and non-P450 phase 1 oxidation enzyme pathways are responsible mostly for the bio activation of relatively inert parent compounds to reactive (toxic/carcinogenic/mutagenic) metabolites. Phase 2 conjugating pathways are responsible mostly for the biotransformation of toxic parent compounds or metabolites to less toxic compounds. Both phase 1 and phase 2 pathways are present in enterocytes. In embodiments, enterocytes can be used to model enteric metabolism for orally ingested toxicants.

In embodiments, the present in vitro reagent—metabolically competent isolated intestinal mucosa comprising villi—are isolated from a variety of genetically diverse individuals who may respond differently to biologic and pharmacologic agents. Genetic diversity can have indirect and direct effects on metabolism of a test compound. In embodiments, the metabolically competent in vitro reagent comprises a pool of isolated intestinal mucosa from multiple individuals or donors. In certain embodiments, the metabolically competent isolated intestinal mucosa comprising villi are reflective of the heterogeneity of a population of individuals.

In certain embodiments, the in vitro reagent is used for high-throughput screening to test the metabolic effects or response to a range of test compounds. In that instance, the in vitro reagent may be used with a cell culture vessel that is a multi-well plate, such as a 6-well; 12-well; 24-well; 48-well, 96-well; 384-well, 1536-well plate or any combination thereof. In alternative embodiments, the methods use a cell culture vessel with a single assay well.

For screening test compounds using the in vitro reagent for their metabolic effect on the cells, the in vitro reagent comprising metabolically competent isolated intestinal mucosa comprising villi are thawed (or used freshly isolated) and placed in a cell culture vessel with cell culture medium. The term “culture condition” encompasses cells, media, factors, time and temperature, atmospheric conditions, pH, salt composition, minerals, etc. Cell culturing is typically performed in a sterile environment mimicking physiological conditions, for example, at 37° C. in an incubator containing a humidified 92-95% air/5-8% CO₂ atmosphere. In embodiments, the cell culture temperate is a range from 30-45° C. Cell culturing may be carried out in nutrient mixtures containing undefined biological fluids such a fetal calf serum, or media that is fully defined and serum free. A variety of culture media are known in the art and are commercially available.

In embodiments, the in vitro reagent may be cultured for a time period from a few minutes to days prior addition of the test compound. In embodiments, the xenobiotic test compound is placed in the cell culture vessel wherein the in vitro reagent (i.e. cell culture medium admixture comprising isolated intestinal mucosa comprising villi) is then incubated under appropriate cell culture conditions as disclosed herein for a time period of 1 minute to up to 10 days. In embodiments, the incubation period can be at least 1 to 59 minutes, 1 hours, 2 hours, 5 hours, 10 hours, 15 hours, 24 hours, 2 days, 3 days, 4 days, 5 days, 6 days, 7 days, 8 days, 9 days or at least 10 days. In embodiments, the incubation time period is not longer than 1 day, 2 days, 5 days, 6 days, 7 days, 8 days, 9 days or not longer than 10 days. In embodiments, the cell culture conditions replicate physiological conditions as much as possible. The term “physiological conditions” as used herein is defined to mean that the cell culturing conditions are very specifically monitored to mimic as closely as possible the natural tissue conditions for a particular type of cell in vivo.

In embodiments, the xenobiotic test compound is considered an input variable, and is used interchangeably herein with a test compound. The test compounds are screened for biological activity by adding to a pharmacokinetic-based culture system (e.g. present in vitro reagent), and then assessing the metabolically competent cells of the mucosa comprising villi (or culture medium) for changes in output variables of interest, e.g., consumption of O₂, production of CO₂, cell viability, expression of proteins of interest (protein expression), cell function, expression of genes of interest (gene expression), metabolite formation or metabolite profiles. The test compound is typically added in solution, or readily soluble form, to the medium of cells in culture. The test compound can be added using a flow through system, or alternatively, adding a bolus to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall composition of the culture medium should not change significantly with the addition of the bolus, or between the two solutions in a flow through method.

In embodiments, the test compound includes pharmacologically active drugs or drug candidates and genetically active molecules. Test compounds of interest include chemotherapeutic agents, anti-inflammatory agents, hormones or hormone antagonists, ion channel modifiers, and neuroactive agents. Exemplary of pharmaceutical agents suitable for this invention are those described in “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming Organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

In embodiments, the test compound includes all of the classes of molecules disclosed herein and may further or separately comprise samples of unknown content. While many samples will comprise compounds in solution, solid samples that can be dissolved in a suitable solvent may also be assayed. Samples containing test compounds of interest include environmental samples, e.g., ground water, sea water, or mining waste; biological samples, e.g., lysates prepared from crops or tissue samples; manufacturing samples, e.g., time course during preparation of pharmaceuticals; as well as libraries of compounds prepared for analysis; and the like. Samples of interest include test compounds being assessed for potential therapeutic value, e.g., drug candidates from plant or fungal cells.

Test compounds are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, naturally or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification to produce structural analogs.

In embodiments, following incubation of the in vitro reagent with the test compound an end point analysis is performed to determine the effect of the test compound on the metabolically competent cells of the isolated intestinal mucosa comprising villi. In embodiments, the end point analysis identifies the output variable (e.g. the effect of the test compound) of the in vitro reagent. In embodiments, output variables are quantifiable elements of the cells, particularly elements that can be accurately measured in a cell culture system. An output variable can be any cell component or cell product including, e.g., viability, respiration, metabolism, cell surface determinant, receptor, protein or conformational or posttranslational modification thereof, lipid, carbohydrate, organic or inorganic molecule, mRNA, DNA, or a portion derived from such a cell component. In embodiments, the output variable is directly or indirectly a result of the test compound or its metabolite. While most output variables will provide a quantitative readout, in some instances a semi-quantitative or qualitative result will be obtained. Readouts may include a single determined value, or may include mean, median value or the variance. Characteristically a range of readout values will be obtained for each output. Variability is expected and a range of values for a set of test outputs can be established using standard statistical methods.

In embodiments, the end point assay is an Ames Salmonella histidine reversion assay (Ames test) for genotoxicity, a mammalian or non-mammalian genotoxicity assay, a mammalian or non-mammalian pharmacological assay.

Various methods can be utilized for quantifying the presence of selected metabolism markers. Liquid chromatography (LC), mass spectrometry (MS), and their combination (LC/MS-MS) are routinely used for the quantification of metabolites. For non-LC/MS measurement of the amount of a molecule that is present, a convenient method is to label the molecule with a detectable moiety, which may be fluorescent, luminescent, radioactive, or enzymatically active. Fluorescent and luminescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to auto-fluoresce, e.g., by expressing them as green fluorescent protein chimeras inside cells (for a review, See Jones et al. (1999) Trends Biotechnol. 17(12):477-81).

Output variables may be measured by immunoassay techniques such as, immunohistochemistry, radioimmunoassay (RIA) or enzyme linked immunosorbance assay (ELISA) and related non-enzymatic techniques. These techniques utilize specific antibodies as reporter molecules that are particularly useful due to their high degree of specificity for attaching to a single molecular target. Cell based ELISA or related non-enzymatic or fluorescence-based methods enable measurement of cell surface parameters. Readouts from such assays may be the mean fluorescence associated with individual fluorescent antibody-detected cell surface molecules or cytokines, or the average fluorescence intensity, the median fluorescence intensity, the variance in fluorescence intensity, or some relationship among these. For toxicity assays, outputs can include measurement of cell viability such as enzyme release, cellular ATP contents, reactive oxygen species formation, decrease of reduced glutathione, protein synthesis, protein contents, DNA contents, dye exclusion, dye inclusion, and cell detachment. For pharmacological assays, specific disease target related assays can be used. For genotoxicity assays, endpoints measured may include DNA damage, chromosomal aberration, mutant generation, and induction of DNA repair.

In embodiments, the results of screening assays may be compared to results obtained from a reference compound, concentration curves, controls (with and without metabolically competent cells), etc. The comparison of results is accomplished by the use of suitable deduction protocols, AI (artificial intelligence or machine learning) systems, statistical comparisons, etc.

A database of reference output data can be compiled. These databases may include results from known agents or combinations of agents, as well as references from the analysis of cells treated under environmental conditions in which single or multiple environmental conditions or parameters are removed or specifically altered. A data matrix may be generated, where each point of the data matrix corresponds to a readout from an output variable, where data for each output may come from replicate determinations, e.g., multiple individual cells of the same type.

The readout may be a mean, average, median or the variance or other statistically or mathematically derived value associated with the measurement. The output readout information may be further refined by direct comparison with the corresponding reference readout. The absolute values obtained for each output under identical conditions will display a variability that is inherent in live biological systems and also reflects individual cellular variability as well as the variability inherent between individuals.

The following examples are put forth so as to provide those of ordinary skill in the art with a complete disclosure and description of how to use the embodiments provided herein and are not intended to limit the scope of the disclosure nor are they intended to represent that the Examples below are all of the experiments or the only experiments performed. Efforts have been made to ensure accuracy with respect to numbers used (e.g. amounts, temperature, etc.) but some experimental errors and deviations should be accounted for. Unless indicated otherwise, parts are parts by volume, and temperature is in degrees Centigrade. It should be understood that variations in the methods as described can be made without changing the fundamental aspects that the Examples are meant to illustrate.

### EXAMPLES

The following examples are put forth so as to provide those of ordinary skill in the art with a complete disclosure and description of how to use the embodiments provided herein and are not intended to limit the scope of the disclosure nor are they intended to represent that the Examples below are all of the experiments or the only experiments performed. Efforts have been made to ensure accuracy with respect to numbers used (e.g. amounts, temperature, etc.) but some experimental errors and deviations should be accounted for. Unless indicated otherwise, parts are parts by volume, and temperature is in degrees Centigrade. It should be understood that variations in the methods as described can be made without changing the fundamental aspects that the Examples are meant to illustrate.

### Example 1: Isolation of Intestinal Mucosa

Isolation of intestinal mucosal comprising villi are eluted using methods known in the art. Those methods include elution of the intestinal mucosa from the lumen of intestines by: flowing cold vulture medium through or over the lumen; adding an enzyme (e.g. collagenase; protease) to the lumen, followed by incubation at 37° C., wherein the mucosa comprising villi are released with a culture medium wash; or, by adding a chelating agent, such as EDTA, to the lumen wherein the mucosa comprising villi are released following an incubation period at 37° C.

In embodiments, the human small intestine was dissected, cut longitudinally, and washed briefly with ice-cold Dulbecco's Modified Eagle's Minimal Medium (DMEM). The tissue was further dissected into smaller pieces and incubated in DMEM at 4° C. with gentle shaking to release the intestinal mucosa. The released intestinal mucosa was collected as a pellet by centrifugation at 100×g for 10 minutes. The pellet was resuspended in an appropriate medium for the intended use, including cryopreservation and metabolism assays. See FIG. 1.

In other embodiments, upon collagenase digestion, the intestinal mucosa detached from the intestine as large sheets consisting mainly of intestinal villi. The sheets of intestinal mucosal epithelia were gently homogenized into small, multicellular fragments before cryopreservation. The light homogenization was necessary to allow the cryopreserved intestinal mucosa suspension to be delivered with a multichannel pipet into the wells of 96-well plates for experimentation. See FIG. 2.

### Example 2: Cryopreservation of Isolated Intestinal Mucosa; Preparation of an In Vitro Reagent for Evaluating Biological Activity of a Test Substance

In embodiments, the isolated intestinal mucosa suspension comprising villi was subjected to cryopreservation. The isolated intestinal mucosal comprising villi were prepared according to Example 1. A cryo-preservant, dimethyl sulfoxide (DMSO) was added slowly to the mucosa suspension at 4 deg. C. until the final concentration reaches 10% of the final total volume (addition of 100 mL of DMSO to 900 mL of mucosa suspension). The suspension was dispensed into cryovials (e.g. 1 mL per vial) and cryopreserved in a programmable cryofreezer at a freezing rate of −1 deg. C. per minute until −95 deg. C. The cryovials were stored in liquid nitrogen until use. See FIG. 2.

### Example 3: Cryopreserved Isolated Intestinal Mucosa Retain Drug Metabolizing Activity

In embodiments, isolated intestinal mucosa comprising villi were prepared according to Example 2 and demonstrated to retain their drug metabolizing activity.

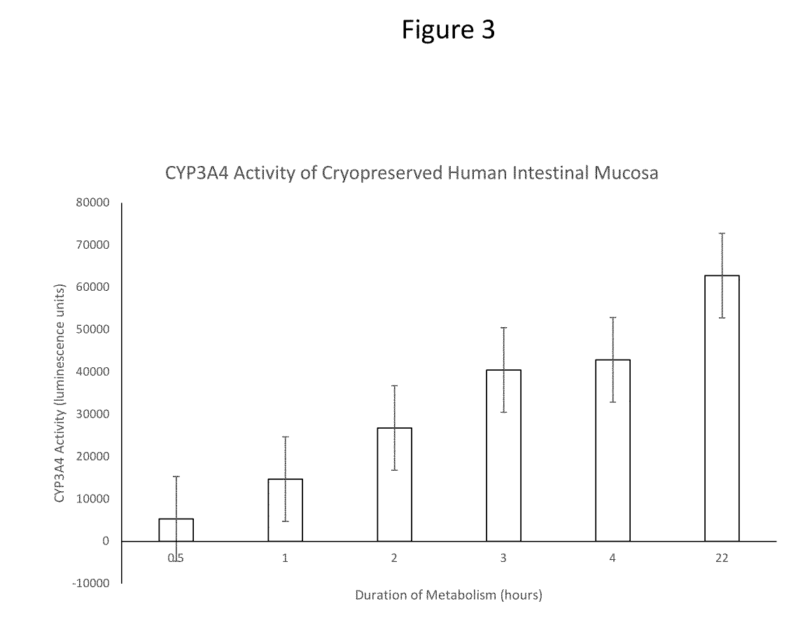

The major drug metabolizing enzyme of the intestinal mucosa is cytochrome P450 isoform 3A4 (CYP3A4). Incubation of cryopreserved human intestinal mucosa with a CYP3A4 substrate, luciferin IPA led to time-dependent increases in metabolite (luciferin) formation quantified by luminescence. The results showed that the cryopreserved human intestinal mucosa was capable of drug metabolism. See FIG. 3.

### Example 4: Freshly Isolated Intestinal Mucosa Retain Drug Metabolizing Activity

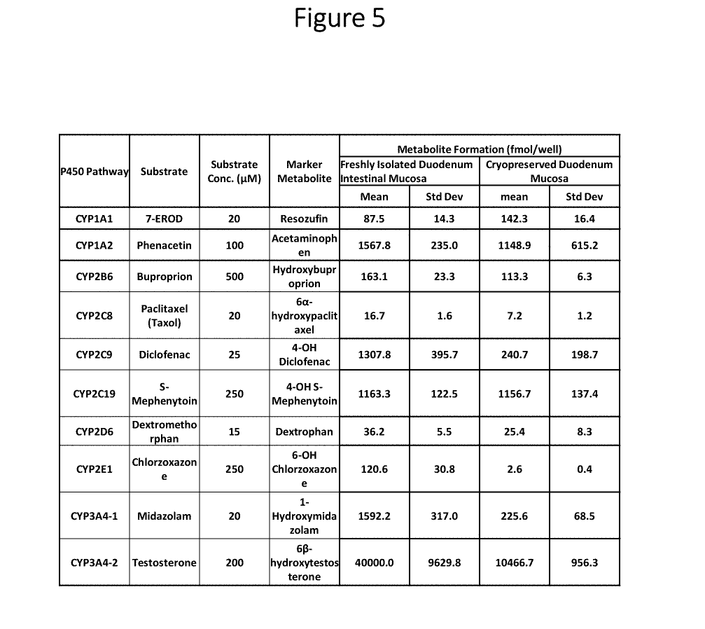

In embodiments, isolated intestinal mucosa comprising villi were prepared according to Example 1 and demonstrated to retain their drug metabolizing activity. Freshly isolated intestinal mucosa was suspended in a protein free incubation medium (Hepatocyte Enterocyte Incubation Medium, In Vitro ADMET Laboratories, Columbia, Md.). Aliquots of 50 uL of the mucosa suspension were added to wells of a 96-well plate containing 50 uL of drug metabolizing enzyme substrates (See FIG. 5) at 2× of the intended final concentration. The plate was incubated at 37 deg. C. for an intended time period (e.g. 30 min.) after which 100 uL aliquots of acetonitrile was added to each well to terminate the reaction. The 96-well plate was then stored at −80 deg. C. For the quantification of metabolite formation, the plate was thawed at room temperature. The samples were quantified for metabolite formation using liquid chromatography mass spectrometry. Results are shown in FIG. 5, which demonstrate the isolated intestinal mucosa comprising villi retain their drug metabolizing activity after being isolated from the lumen of the intestine.

FIG. 5 shows retention of drug metabolizing enzyme activities in freshly isolated (Example 3) and cryopreserved (Example 4) intestinal mucosa. P450-dependent drug metabolism is the most important pathway for drug metabolism. The results show that both freshly isolated and cryopreserved intestinal mucosa retained drug metabolizing enzyme activities

### Example 5: Cryopreserved Isolated Intestinal Mucosa Retain Drug Metabolizing Activity

In embodiments, isolated intestinal mucosa comprising villi were prepared according to Example 2 and demonstrated to retain their drug metabolizing activity. A vial of cryopreserved intestinal mucosa was thawed in a 37 deg. C. water bath and added to 50 mL of culture medium (Universal Cryopreservation Plating Medium, In Vitro ADMET Laboratories, Columbia, Md.). The recovered mucosa pellet was suspended in a protein free incubation medium (Hepatocyte Enterocyte Incubation Medium, In Vitro ADMET Laboratories, Columbia, Md.). Aliquots of 50 uL of the mucosa suspension were added to wells of a 96-well plate containing 50 uL of drug metabolizing enzyme substrates (See FIG. 5) at 2× of the intended final concentration. The plate was incubated at 37 deg. C. for an intended time period (e.g. 30 min.) after which 100 uL aliquots of acetonitrile were added to each well to terminate the reaction. The 96-well plate was then stored at −80 deg. C. For the quantification of metabolite formation, the plate was thawed at room temperature. The samples were quantified for metabolite formation using liquid chromatography mass spectrometry. Results are shown in Table 5, which demonstrate the cryopreserved isolated intestinal mucosa comprising villi retain their drug metabolizing activity after being isolated from the lumen of the intestine and frozen.

### Example 6: Application of Cryopreserved Isolated Intestinal Mucosa in the Evaluation of Intestinal Drug Toxicity

In embodiments, isolated intestinal mucosa comprising villi were prepared according to Example 1 or Example 2 and are used herein in methods for evaluating biological activity of a test substance, including metabolism, toxicity, genotoxicity, carcinogenicity, drug-drug interactions, receptor binding, receptor inhibition, biochemical function, gene expression, protein expression, or pharmacological activities.

A vial of cryopreserved intestinal mucosa was thawed in a 37 C deg. water bath and added to 50 mL of culture medium (Universal Cryopreservation Plating Medium, In Vitro ADMET Laboratories, Columbia, Md.). The recovered mucosa pellet was suspended in a protein free incubation medium (Hepatocyte Enterocyte Incubation Medium, In Vitro ADMET Laboratories, Columbia, Md.). Aliquots of 50 uL of the mucosa suspension were added to wells of a 96-well plate containing 50 uL of an enterotoxic drug, naproxen, at 2× of the intended final concentration. The plate was incubated at 37 deg. C. for 24 hrs after which cell viability was determined by the quantification of cellular ATP contents (ATPLite, Perkin Elmers). Results are shown in Table 1.

TABLE 1
Application of cryopreserved human intestinal mucosa in
the evaluation of drug-induced enterotoxicity. A known enterotoxic
drug, naproxen, was evaluated. Results show dose-dependent
decrease in viability, demonstrating that the cryopreserved
human intestinal mucosa can be applied towards the evaluation
of enterotoxic potential of drugs.

| | |
|---|---|
| Naproxen Concentration | Relative Viability |

| (mg/mL) | mean | sd |
|---|---|---|
| 0 | 100.00 | 16.27 |
| 0.156 | 72.27 | 4.10 |
| 0.312 | 67.51 | 9.52 |
| 0.625 | 43.94 | 8.65 |
| 1.25 | 19.79 | 3.13 |
| 2.5 | 7.74 | 0.69 |
| 5 | 2.71 | 0.45 |
| 10 | 1.19 | 0.08 |

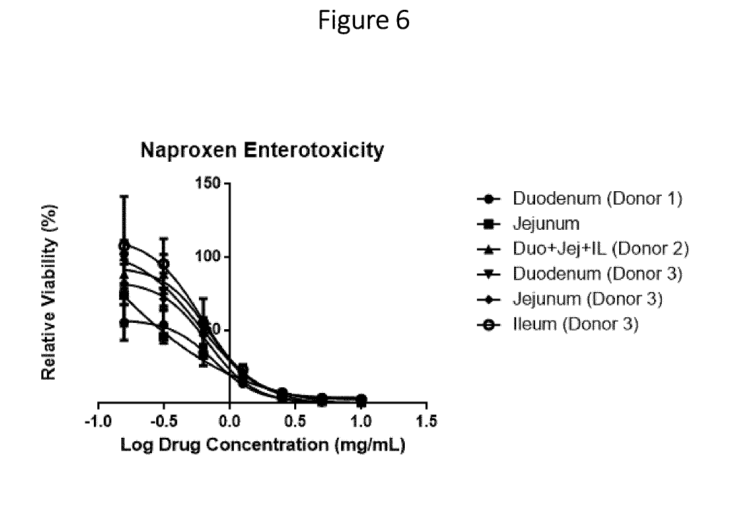

The above experiment was repeated with isolated intestinal mucosa comprising from three different sections of the intestine (duodenum, jejunum and ileum), wherein the isolated intestinal mucosa was prepared from three donors according to Example 1. See FIG. 6.

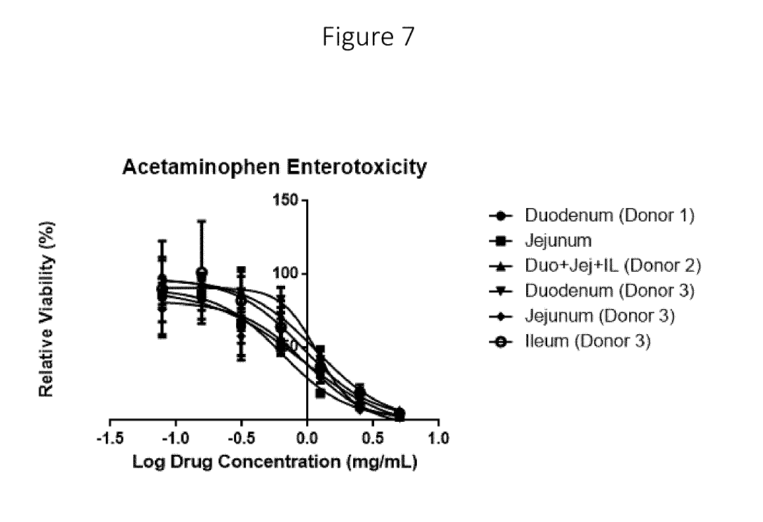

The above experiment was further repeated with acetaminophen in place of naproxen. See FIG. 7. Results from both FIGS. 6 and 7 show a dose-dependent decrease in viability, demonstrating that the isolated human intestinal mucosa comprising villi can be applied towards the evaluation of enterotoxic potential of drugs.

### Example 7: Induction of P450 in Cryopreserved Isolated Intestinal Mucosa Comprising Villi

In embodiments, isolated intestinal mucosa comprising villi were prepared according to Example 2 and demonstrated utility in screening for drug-drug interactions of test compounds. Freshly isolated and cryopreserved human mucosa will be cultured in the presence and absence of a test article, followed by addition of pathway-specific substrates of key drug metabolizing enzyme activities followed by quantification of metabolite formation. Inhibition or induction of drug metabolizing enzyme activities will indicate potential pharmacokinetic drug interactions with co-administered drugs that are substrates of the affected pathways. An example is the evaluation of induction of intestinal mucosal CYP3A4 activity, a key drug metabolizing enzyme for orally administered drug and a key determinant of oral bioavailability.

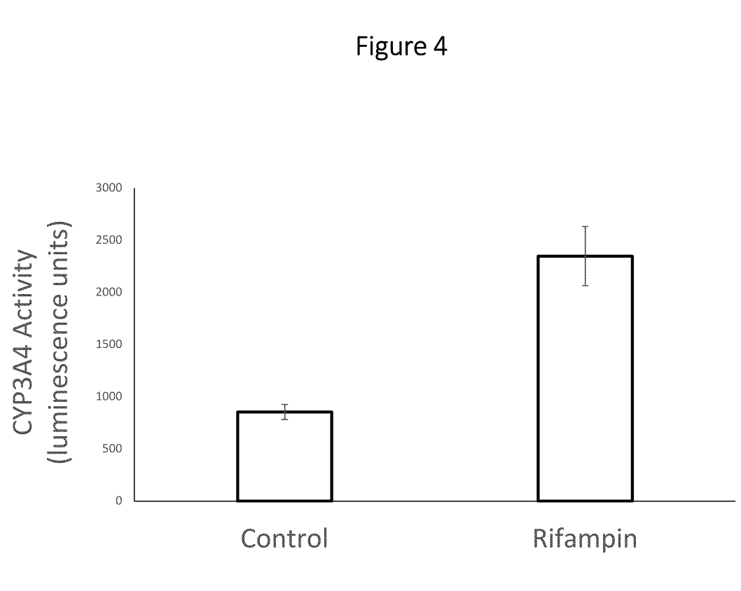

Rifampin is known to induce human intestinal CYP3A4 activity (cytochrome P45 isoform 3A4), leading to significant drug-drug interaction exhibited by accelerated metabolism of co-administered drugs that are CYP3A4 substrates. Treatment of cryopreserved human intestinal mucosa with rifampin (20 uM) led to enhancement of CYP3A4 activities. See FIG. 4. This experiment demonstrated that cryopreserved human intestinal mucosa can be used as an experimental system to evaluate this important mechanism of drug-drug interactions. At present, there are no in vitro experimental systems for the evaluation of intestinal P450 induction.

### Example 8: Cryopreserved Isolated Intestinal Mucosa Comprising Villi Retain P450 and Non-P450 Drug Metabolic Enzyme Activities Characteristic of the Small Intestine

Provided herein is an in vitro intestinal experimental system wherein isolated intestinal mucosa from human small intestines can be isolated and cryopreserved as multicellular fragments to retain viability and functions. The thawed cryopreserved human intestinal mucosa were found to exhibit P450 and non-P450 drug metabolizing enzyme activities, were responsive to the enterotoxicity of acetaminophen and naproxen, and showed robust (approx. 300-fold) induction of CYP24A1 transcription by vitamin D3 and moderate (approx. 3-fold) induction of CYP3A4 transcription by vitamin D3 and rifampin. This example demonstrates that isolation and cryopreservation of intestinal mucosa from the duodenum, jejunum, and ileum of human intestines retain drug metabolism activity, responsiveness to P450 inducers and gastrointestinal toxicants.

In embodiments, isolated intestinal mucosa comprising villi were prepared according to Example 1 and Example 2. The isolated epithelial consist mainly of individual villi. These relatively large mucosal fragments were gently homogenized to form smaller, multicellular fragments followed by cryopreservation. The morphology of the freshly isolated villi and the multicellular fragments of the thawed CHIM are shown in FIG. 2

The cryopreserved vials of the isolated intestinal mucosa comprising villi were removed from liquid nitrogen storage and thawed in a 37° C. water bath for approximately 2 min. The contents of each individual vial were decanted into a 50 ml conical tube containing Cryopreserved Enterocyte Recovery Medium, (In Vitro ADMET Laboratories, Columbia, Md.) that was pre-warmed in a 37° C. water bath. The thawed in vitro reagent was recovered by centrifugation at 100×g for 10 min at room temperature. After centrifugation, the supernatant was removed by decanting. A volume of 5 mL of 4° C. Hepatocyte/Enterocyte Incubation Medium, (In Vitro ADMET Laboratories, Columbia, Md.) was added to the intact pellet of enterocytes at the bottom of the conical tube followed by briskly re-pipetting 5 times with a P1000 micropipet to create an even suspension of the intestinal mucosal fragments.

Substrates, concentrations, and the metabolites quantified for the multiple drug metabolism pathways evaluated are shown in Table 2 for P450 isoforms and Table 3 for non-P450 drug metabolizing enzymes.

TABLE 2
A summary of the P450 isoform-selective substrates, substrate
concentrations, and their respective metabolites to be
quantified for evaluation of the drug metabolizing enzyme
activities of the cryopreserved intestinal mucosa.

| | | Substrate | |
|---|---|---|---|
| | | Conc. | Metabolites |
| Isoform | Substrate | (μM) | Quantified |
| CYP1A1 | 7-Ethoxyresorufin | 20 | Resozufin |
| CYP1A2 | Phenacetin | 100 | Acetaminophen |
| CYP2A6 | Coumarin | 50 | 7-HC, 7-HC-Sulfate, |
| | | | 7-HC-Glucuronide |
| CYP2B6 | Bupropion | 500 | Hydroxybupropion |
| CYP2C8 | Paclitaxel | 20 | 6α-hydroxypaclitaxel |
| CYP2C9 | Diclofenac | 25 | 4-hydroxydiclofenac |
| CYP2C19 | S-Mephenytoin | 250 | 4-hydroxy |
| | | | S-Mephenytoin |
| CYP2D6 | Dextromethorphan | 15 | Dextrophan |
| CYP2E1 | Chlorzoxazone | 250 | 6-hydroxy chlorzoxazone |
| CYP3A4/5-1 | Midazolam | 20 | 1-hydroxymidazolam |
| CYP3A4/5-2 | Testosterone | 200 | 6β-hydroxytestosterone |
| CYP2J2 | Astemizole | 50 | O-Demethyl Astemizole |

TABLE 3
A summary of the non-P450 pathway selective substrates
and their respective metabolites quantified in
cryopreserved intestinal mucosa.

| | | Substrate | |
|---|---|---|---|
| DME | | Conc. | |
| Pathway | Substrate | (μM) | Metabolites Quantified |
| ECOD | 7-Ethoxycoumarin | 100 | 7-HC, 7-HC-Sulfate, |
| | | | 7-HC-Glucuronide |
| UGT | 7-Hydroxycoumarin | 100 | 7-Hydroxycoumarin |
| | | | Glucuronide |
| SULT | 7-Hydroxycoumarin | 100 | 7-Hydroxycoumarin |
| | | | Sulfate |
| FMO | Benzydamine HCl | 250 | Benzydamine-N-Oxide |
| MAO | Kynuramine HBr | 160 | 4-hydroxyquinoline |
| AO | Cabazeran | 20 | 4-Hydroxycabazeran |
| NAT1 | 4-Aminobenzoic | 200 | N-Acetyl-p-aminobenzoic |
| | Acid | | acid |
| NAT2 | Sulfamethazine | 100 | N-Acetyl-sulfamethazine |
| CES2 | Irinotecan | 50 | SN38 |

Determination of drug metabolizing enzyme activities of the intestinal mucosa was performed via incubation with metabolism substrates in a cell culture incubator maintained at 37° C. with a humidified atmosphere of 5% CO₂. A volume of 50 μL of drug metabolizing enzyme substrates at 2× of the final desired concentrations was added into the designated wells of a 96 well plate (reaction plate). The reaction plate was placed in a cell culture incubator for 15 minutes to prewarm the substrate solutions to 37° C., followed by addition of metabolically competent isolated intestinal mucosa comprising villi at a volume of 50 μL per well to initiate the reaction. The reaction plates were then incubated at 37° C. for 30 minutes. All incubations were performed in triplicate. Metabolism was terminated in each well by the addition of 200 μl acetonitrile containing 250 nM of the internal standard tolbutamide. The incubated samples were stored at −80° C. for the subsequent LC/MS-MS analysis.

Gene transcription in the intestinal mucosa was quantified based on reverse transcription-real time PCR (RT-PCR) using the 7500 Fast Real-Time PCR system (Applied Biosystems, Foster City, Calif.). Total RNA was extracted using the mini RNeasy kit (QIAGEN, www.qiagen.com) according to instructions provided by the manufacturer for isolation of total RNA from the intestinal mucosa. At first, reverse transcription (RT) was performed with approximately 200 ng of isolated RNA using the High Capacity cDNA Reverse Transcription Kit (Thermo Fisher Scientific Inc., www.thermofisher.com). Subsequently, quantitative PCR analysis was performed on RT reactions using gene specific primer/probe sets and the Taqman Fast Universal PCR Master Mix (Thermo Fisher). The relative quantity of the target gene was compared with that of the reference transcription of glucose 6-phosphate dehydrogenase (GAPDH) as determined by the AACT method and as previously described (Fahmi, Kish et al. 2010).

In vitro cytotoxicity evaluation with cryopreserved intestinal mucosa was performed in 96-well plates. After recovery from cryopreservation as described above, the CHIM pellet was resuspended in 5 mL of Hepatocyte Incubation Medium (IVAL, Columbia, Md.). The cryopreserved intestinal mucosa suspension was added to each well of the 96-well plates followed by addition of 50 μL of the toxicants (acetaminophen and naproxen) at 2× of the final desired concentration. The cryopreserved intestinal mucosa cultures were then incubated in a CO₂ cell culture incubator kept at 37° C. in a highly humidified atmosphere of 5% CO₂. After an incubation duration of 24 hrs., cell viability was determined based on cellular ATP contents as previously described. Results are presented as relative viability which is a ratio of the cellular contents of treated cultures to that of solvent control cultures.

As cryopreserved intestinal mucosa consists of multiple cell aggregates, cellular contents were quantified as protein concentrations. Determination of protein concentration was performed using the Pierce BCA Protein Assay Kit (Thermo Fisher Scientific, Waltham, Mass.) per instructions specified by the manufacturer.

Quantification of metabolites formed upon incubation of the cryopreserved isolated intestinal mucosa with various substrates was performed using liquid chromatography tandem mass spectrometry (LC/MS-MS). Upon thawing, the samples were centrifuged at 3,500 rpm for 5 minutes. An aliquot of 100 μL of supernatant from each was transferred to a 96 well plate and was diluted with 200 μL of deionized water with mixing before LC/MS/MS analysis. CYP1A2, CYP2A6, CYP2B6, CYP2C8, CYP2C9, CYP2C19, CYP2D6, CYP2E1, CYP3A4 (midazolam 1′-hydroxylation), CYP3A4 (testosterone 6β-hydroxylation), ECOD, UGT, SULT, FMO, MAO, AO, and NAT2 metabolites, as well as acetaminophen metabolism were quantified performed by using API 5000 mass spectrometer with an electrospray ionization source (AB SCIEX, Framingham, Mass.) connected to Waters Acquity UPLC (Waters Corporation, Milford, Mass.) using LC/MS/MS MRM mode, monitoring the mass transitions (parent to daughter ion) as previously described (Ho, Ring et al. 2017). An Agilent Zorbax Eclipse Plus C18 column (4.6×75 mm i.d., 3.5 μm; Agilent Technologies, Santa Clara, Calif.) at a flow rate of 0.7 mL/min was used for the chromatography separation. The mobile phase consisted of 0.1% formic acid in water (A) and 0.1% formic acid in acetonitrile (B). The gradient for the positive ion mode operation was programmed as: 0 to 1 min, increased B from 5 to 90%; 1 to 1.5 min, keep B at 90%; 1.5 to 1.75 min, decreased B to 5%; run-time 3 min. The gradient program for the negative ion mode was: 0 to 2.5 min, increase B from 30 to 95%; 2.5 to 3.0 min, keep B at 95%; 3 to 3.2 min, decrease B to 30%; run-time 4 min. For conjugates, the gradients and run time may be adjusted for better separation. Data acquisition and data procession were performed with the software Analyst 1.6.2 (AB SCIEX, Framingham, Mass.).

Data are presented as mean and standard deviation of triplicate incubations derived using the Microsoft Excel 6.0 software. Statistical analysis was performed using student's t-test with the Microsoft Excel 6.0 software, with the probability of p<0.05 to be considered statistically significant. Specific activity (pmol/min/mg protein) of each drug metabolizing enzyme pathway was determined by dividing the total metabolite formed by the incubation time and normalized to protein contents. Graphpad Prism 6.0 was used for the determination of EC50 for the cytotoxicity of acetaminophen and naproxen.

P450 Isoform Drug Metabolizing Enzyme Activities in Cryopreserved Isolated Intestinal Mucosa.

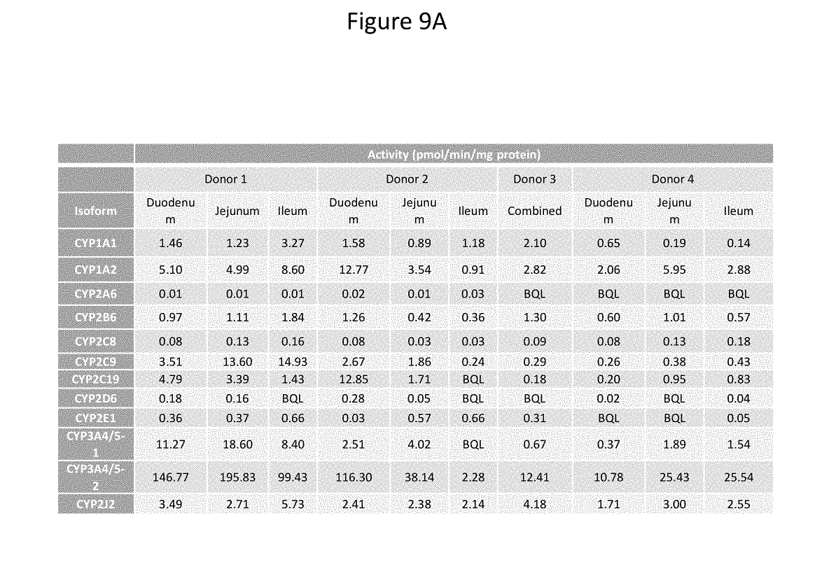

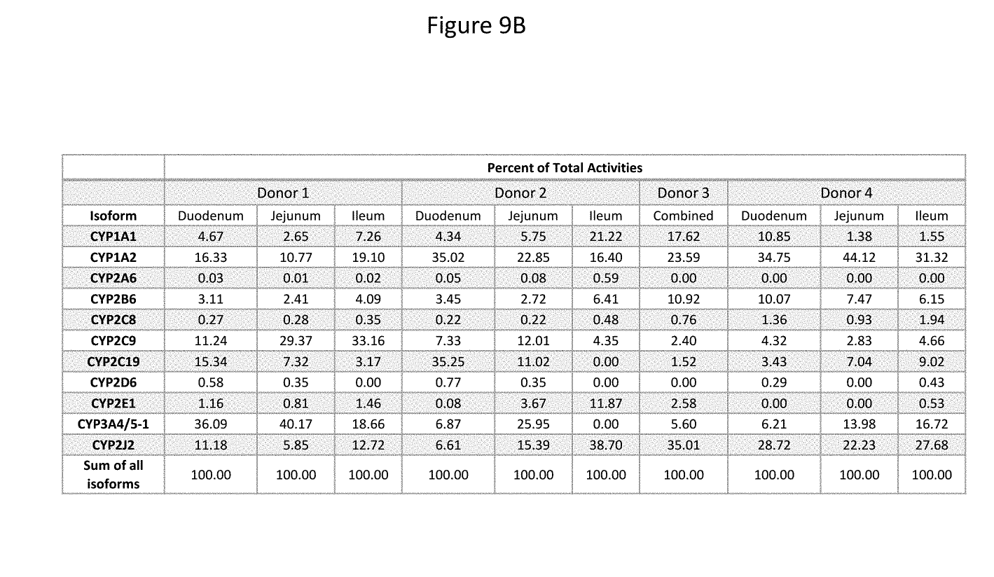

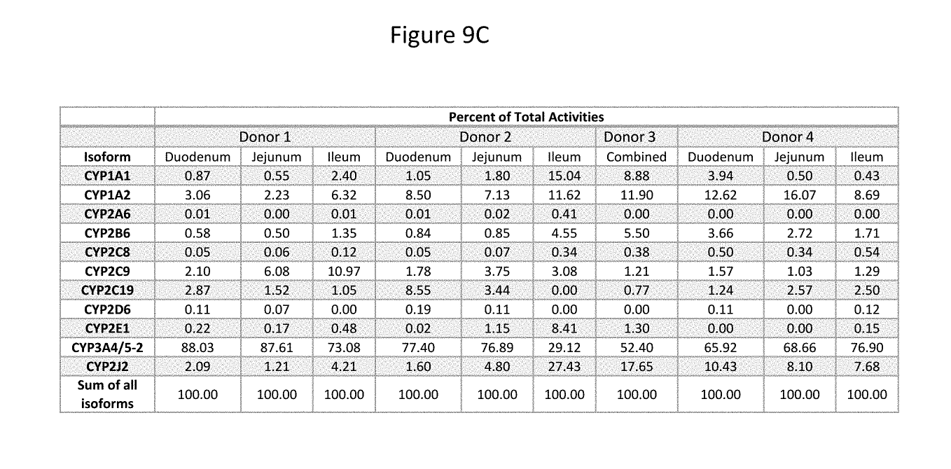

CYP1A1, CYP1A2, CYP2A6, CYP2B6, CYP2C8, CYP2C9, CYP2C19, CYP2D6. CYP2E1, CYP3A4 and CYP2J2 activities quantified in cryopreserved intestinal mucosa using isoform-selective substrates are shown in FIG. 9A. The highest activity was observed for CYP3A4 measured as testosterone 6-b hydroxylation. See FIG. 9C. CYP2C9 and CYP2C19 activities were higher than that observed for CYP2A6, CYP2B6, CYP2C8, CYP2D6, and CYP2E1. The relative distribution of the P450 isoform activities is shown in FIGS. 9B and 9C, wherein distribution of P450 isoform activities in cryopreserved intestinal mucosa using midazolam-1′hydroxylation (FIG. 9B) and testosterone 6b-hydroxylation (FIG. 9C) as CYP3A4 activity. Results are expressed as percent of the arithmetic sum of the specific activities (pmol/min/mg protein) of the P450 isoforms evaluated.

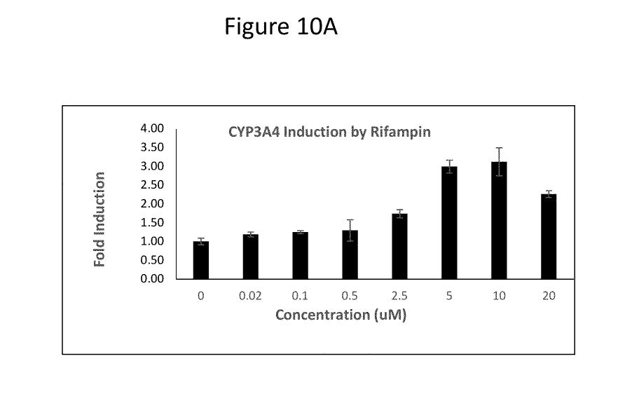

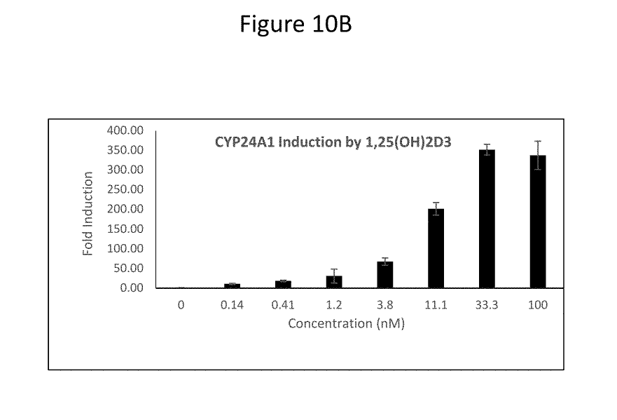

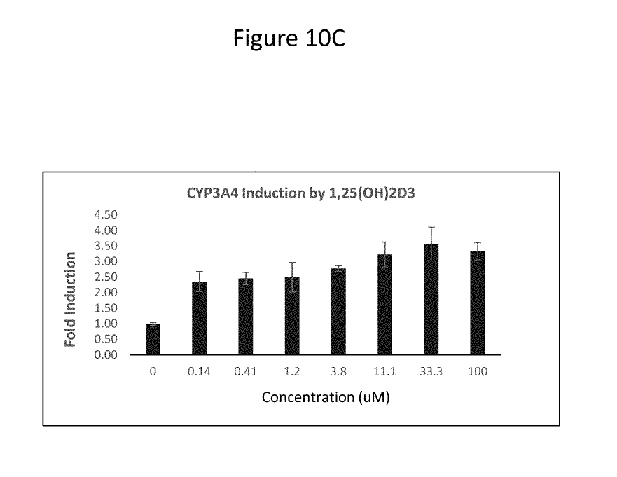

P450 induction in cryopreserved metabolically competent isolated intestinal mucosa was evaluated based on gene transcription. Following a 24-hr treatment period, dose dependent induction of CYP3A4 mRNA by rifampin and 25-hydroxyD3; and, CYP24A1 mRNA by 25-hydroxyD3 were observed. See FIGS. 10A and 10B. For CYP3A4, the maximal fold of induction was approximately 3-fold for both inducers. Data shown for 25-hydroxyD3 in FIG. 10C. Approximately 300-fold induction of CYP24A1 mRNA was observed for 25-hydroxyD3. See FIG. 10B.

Non-P450 Isoform Drug Metabolizing Enzyme Activities in Cryopreserved Isolated Intestinal Mucosa.

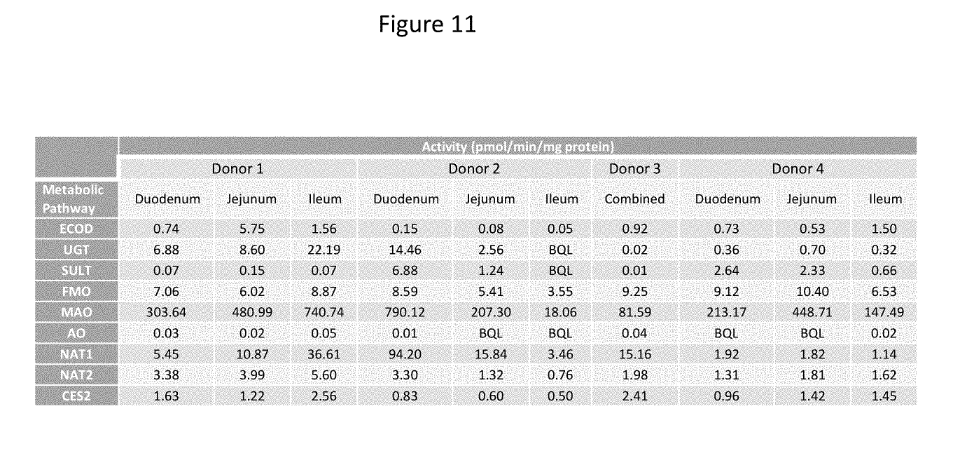

The non-P450 drug metabolizing enzyme pathways evaluated included ECOD, UGT, SULT, FMO, MAO, AO, NAT1, NAT2 and CES2. Results are shown in FIG. 11, wherein Non-P450 drug metabolizing enzyme activities of cryopreserved intestinal mucosa isolated from the duodenum, jejunum and ileum of Donor 1, Donor 2 and Donor 4, and from the entire small intestine (from duodenum to ileum) of Donor 3 are demonstrated (BQL: below limits of quantification). Quantifiable activities were observed for all pathways evaluated except for AO. MAO found to have the highest activity which ranged from similar to higher than that observed for CYP3A4 (testosterone 6b-hydroxylation).

In Vitro Enterotoxicity Assay with Cryopreserved Intestinal Mucosa.

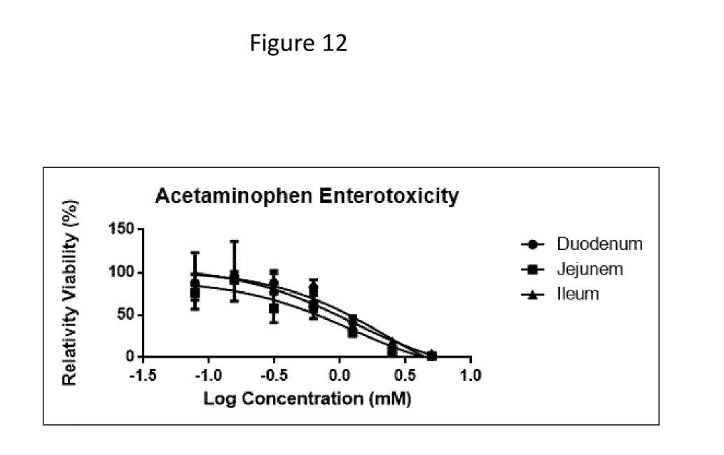

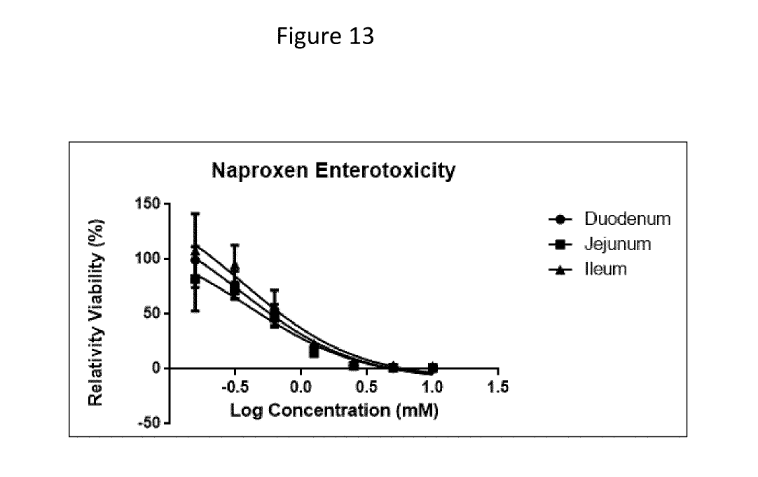

Dose-dependent cytotoxicity was observed for both acetaminophen (FIG. 12) and naproxen (FIG. 13) in cryopreserved intestinal mucosa isolated from the three donors. The calculated IC₅₀ values are shown in Table 4. Results show that naproxen consistently demonstrated higher enterotoxicity than acetaminophen as demonstrated by the lower IC₅₀ values for all cryopreserved intestinal mucosa lots evaluated (Table 4).

TABLE 4
IC50 values for acetaminophen (APAP) and naproxen in cryopreserved
intestinal mucosa isolated from human duodenum, jejunum, and ileum
of Donor 1; duodenum and jejunum of Donor 2, and the combination of all
three small intestine regions of Donor 3. Asterisks represent IC₅₀ values
obtained from naproxen that are statistically significant to be
different (p < 0.05) from that obtained for APAP. IC₅₀ ratio is
calculated by dividing the IC₅₀ for APAP by that for naproxen.
The results show that naproxen was consistently more cytotoxic
than APAP for all three regions of the small intestine.

| | IC₅₀ (mM) | | |
|---|---|---|---|
| Drug | Duodenum | Jejunum | Ileum |
| APAP | 2.25 | 1.22 | 1.20 |
| Naproxen | 0.35* | 0.39* | 0.36* |
| IC₅₀ Ratio | 6.36 | 3.09 | 3.35 |

The present cryopreserved isolated intestinal mucosa comprising villi were found to retain P450 and non-P450 drug metabolic enzyme activities characteristic of the small intestine. As reported before by others using intestinal microsomes (Paine, Khalighi et al. 1997; Perloff, Von Moltke et al. 2003; Yang, Tucker et al. 2004), and by us in cryopreserved enterocytes (Ho, Ring et al. 2017), the major P450 isoform activities were contributed by CYP3A4, especially for testosterone 6b-hydroxylation which was approximately 10 fold higher than that observed for midazolam 1′-hydroxylation. CYP1A2, CYP2C9, CYP2C19 and CYP2J2 represent the non-CYP3A isoforms with substantial activities. CYP1A1 activity was in general lower than that for CYP1A2 and similar to CYP2B6. Minimal, near undetectable activities were observed for CYP2A6, CYP2D6 and CYP2E1. This overall relative distribution of CYP450 isoform activities was similar for all three regions of the small intestine.

For the non-P450 drug metabolizing enzyme activities, the highest was observed for MAO, with activities for all four donors higher than that observed for CYP3A4 quantified by testosterone 6b-hydroxylation. UGT, FMO, NAT1, NAT2 and CES2 activities were also in abundance, similar than that observed for CYP2C9. AO activities were mostly undetectable.

An exciting discovery is that P450 induction could be observed in the present cryopreserved isolated intestinal mucosa comprising villi, thereby representing the only reported P450 induction system with primary enterocytes. Following 24-hour incubation with 1,25(OH)D3, dose-dependent induction of CYP24A1 transcription was observed, with an approximately 300-fold induction. CYP24A1 is the cytochrome P450 component of the 25-hydroxyvitamin D3-24-hydroxylase enzyme that catalyzes the conversion of 25-hydroxyvitamin D3 (25-OH-D3) and 1,25-dihydroxyvitamin D3 (1,25-(OH)2D3) into 24-hydroxylated products, which constitute the degradation of the vitamin D molecule. Dose-dependent induction of CYP3A4 by both 1,25(OH)D3 and rifampin was observed, with a maximum approximately 3-fold induction.

Rifampin and 1,25(OH)D3 induction of CYP3A4 via VDR and PXR pathway has been previously reported in small intestinal cell lines (Kolars, Schmiedlin-Ren et al. 1992; Thummel, Brimer et al. 2001; Thompson, Jurutka et al. 2002; Zheng, Wang et al. 2012) and in small bowel biopsies of patients treated with rifampin in vivo (Kolars, Schmiedlin-Ren et al. 1992). CYP3A induction in the small intestines in vivo may have physiological consequences including enhanced metabolism of vitamin D as well as orally-administered drugs that are substrates of CYP3A4 and CYP24A1. In the application of hepatocyte for the evaluation of hepatic P450 induction, mRNA is found to be a relevant in vitro endpoint that allows the estimation of in vivo effects (e.g., decrease in plasma T1/2 and plasma AUC) (Fahmi, Boldt et al. 2008; Youdim, Zayed et al. 2008; Fahmi, Kish et al. 2010; Einolf, Chen et al. 2014). Our results with mRNA in cryopreserved isolated intestinal mucosa comprising villi, therefore, may be used similarly in the estimation of in vivo enteric metabolic clearance.

Enterotoxicity is a known adverse effect of orally administered drugs. NSAIDS, for instance, are known to cause upper gastrointestinal tract damages (Biour, Blanquart et al. 1987; Semble and Wu 1987). As drug metabolism is a key determinant of toxicity due to metabolic activation and detoxification, an in vitro enteric system with drug metabolism capacity similar to that in the gastrointestinal tract in vivo would be ideal for the early evaluation of gastrointestinal toxicity in drug development. We therefore validated the present in vitro reagent using two NSAIDs known to be associated with gastrointestinal toxicity, acetaminophen (Rainsford and Whitehouse 2006) and naproxen (Curtarelli and Romussi 1973). Both acetaminophen and naproxen have been associated with upper gastrointestinal bleeding and perforations. While intestinal gastrointestinal ulcerations are commonly associated with acid reflux and h. pylori infection, enteropathy has also been associated with enterocyte cytotoxicity. Cryopreserved isolated intestinal mucosa comprising villi may represent a physiological relevant experimental system for the evaluation of cytotoxicity-related enteropathy.

Treatment of the present in vitro reagent (from the duodenum, jejunum, and ileum of Donor 1) with acetaminophen and naproxen led to dose-dependent decreases in viability quantified by cellular ATP contents. See FIGS. 12 and 13. The IC50 of naproxen (0.35-0.39 mM) was statistically significant to be lower than that for acetaminophen (0.92-1.18 mM), with APAP having an IC50 value 3-6 times that for naproxen. Those results with cryopreserved isolated intestinal mucosa comprising villi are consistent with clinical findings that naproxen has a higher enterotoxicity than acetaminophen (Lewis, Langman et al. 2002).

The data presented herein show that cryopreserved isolated intestinal mucosa comprising villi can be useful in the evaluation of enterotoxic potential of orally administered drugs, especially for drugs that may be activated or detoxified by enteric metabolism like APAP (Laine, Auriola et al. 2009; Jaeschke and McGill 2015; Jiang, Briede et al. 2015; Miyakawa, Albee et al. 2015) and naproxen (Miners, Coulter et al. 1996; Rodrigues, Kukulka et al. 1996; Tracy, Marra et al. 1997). In vitro enteric systems such as the present in vitro reagent should be useful in the assessment of enterotoxic potential which can be used in the assessment of in vivo enterotoxicity upon appropriate PBPK modeling considering key in vivo factors including rate of transit, drug dissolution, and available drug concentration at various regions of the intestinal tract.

Provided herein, in embodiments, is an in vitro experimental system (e.g. cryopreserved isolated intestinal mucosa comprising villi) that can aid evaluation of enteric drug properties. Current in vitro experimental models include Caco-2 cells, IPS-derived intestinal cells which in general are deficient of drug metabolizing enzyme activities, especially the sub-optimal expression of CYP3A (Schmiedlin-Ren, Thummel et al. 1997; Cummins, Jacobsen et al. 2004; Negoro, Takayama et al. 2016), the most important drug metabolizing enzyme for enteric drug metabolism. Intestinal microsomes contain drug metabolizing enzymes associated with the endoplasmic reticulum but lack cytosolic, mitochondrial, nuclear, and plasma membrane-associated drug metabolizing enzymes. The in vitro experimental system provided herein represent practical and physiologically relevant in vitro system with “complete” drug metabolizing enzyme pathways for the evaluation of enteric drug metabolism, akin to the use of cryopreserved hepatocytes for hepatic drug metabolism (Li, Reith et al. 1997; Li 2007; Li 2015).

In certain embodiments, the in vitro system (e.g., cryopreserved isolated intestinal mucosa comprising villi) may be used for the evaluation of additional enteric pharmacology and physiology, especially using transcription as endpoints. For instance, as cryopreserved isolated intestinal mucosa comprising villi contain multiple enteric mucosal cell types, it may be useful for the evaluation of the onset and treatment of inflammatory-related events and diseases such as inflammatory bowel disease (Coste, Dubuquoy et al. 2007).

Those skilled in the art can devise many modifications and other embodiments within the scope and spirit of the presently disclosed inventions. Indeed, variations in the materials, methods, drawings, experiments examples and embodiments described may be made by skilled artisans without changing the fundamental aspects of the disclosed inventions. Any of the disclosed embodiments can be used in combination with any other disclosed embodiment.

The disclosed embodiments, examples and experiments are not intended to limit the scope of the disclosure nor to represent that the experiments below are all or the only experiments performed. Efforts have been made to ensure accuracy with respect to numbers used (e.g., amounts, temperature, etc.) but some experimental errors and deviations should be accounted for. It should be understood that variations in the methods as described may be made without changing the fundamental aspects that the experiments are meant to illustrate.

1. A method of manufacturing metabolically competent isolated intestinal mucosal comprising villi eluted from intestines, comprising:
a) eluting the intestinal mucosal from lumen of the intestines to form isolated intestinal mucosa;
b) suspending the isolated intestinal mucosa in a cell culture medium;
c) adding a cryoprotectant to the cell culture medium to form an in vitro reagent; and,
d) storing the in vitro reagent frozen at a temperature of −10° C. to about −175° C.

2. The method of claim 1, wherein the reagent is stored frozen in liquid nitrogen.

3. The method of claim 1, further comprising use of an enzyme in step a).

4. The method of claim 3, wherein the enzyme is collagenase or a protease.

5. The method of claim 1, wherein the isolated intestinal mucosa comprising villi are mammal.

6. The method of claim 5, wherein the isolated intestinal mucosa comprising villi are human.

7. The method of claim 1, wherein the metabolically competent isolated intestinal mucosa comprises enterocytes.

8. The method of claim 1, wherein the metabolically competent isolated intestinal mucosa comprising villi are pooled from more than one donor.

9. An in vitro reagent for evaluating biological activity of a test substance, wherein the reagent is a cryopreserved mixture comprising:
a) a cell culture medium comprising a cryoprotectant; and,
b) metabolically competent isolated intestinal mucosa comprising villi wherein the intestinal mucosa was eluted from an intestine.

10. (canceled)

11. The reagent of claim 9, wherein the isolated intestinal mucosa comprising villi are human.

12. The reagent of claim 9, wherein the metabolically competent isolated intestinal mucosa comprises enterocytes.

13. The reagent of claim 9, wherein the in vitro reagent is stored frozen at a temperature of −10° C. to about −175° C.

14. The reagent of claim 9, wherein the in vitro reagent is thawed prior to use for evaluating biological activity of a test substance.

15. The reagent of claim 9, wherein the in vitro reagent is provided in a kit and further comprises instructions for evaluating biological activity of a test substance using the reagent.

16. The reagent of claim 9, wherein the metabolically competent isolated intestinal mucosa comprising villi are pooled from more than one donor.

17-33. (canceled)

34. A method for evaluating in vitro inhibition or induction of cytochrome P450 by a test compound that is ingested or administered orally, comprising:
a) providing metabolically competent isolated intestinal mucosa comprising villi wherein the intestinal mucosa was eluted from an intestine and previously cryopreserved;
b) culturing the isolated intestinal mucosa in a cell culture vessel incubated at 30-45° C.;
c) introducing a test compound into the cell culture vessel that is suspected of being a P450 ligand;
d) incubating the test compound for 1 minute to 10 days at 30-45° C.; and,
e) performing an end point assay of the isolated intestinal mucosa or cell culture medium to determine P450 activity.

35. The method of claim 34, wherein the test compound suspected of being a P450 ligand is a substrate of P450.

36. (canceled)

37. (canceled)

38. The method of claim 34, wherein the test compound is a drug or drug candidate selected from the group consisting of an organic compound, an inorganic compound, a hormone, a growth factor, a cytokine, a reception, an antibody, an enzyme, a peptide, a NSAID, an aptomer and a vaccine.

39. The method of claim 38, wherein the drug or drug candidate is added at cytotoxic concentrations for identifying and profiling metabolites.

40. The method of claim 34, wherein complete phase 1 oxidation and phase 2 conjugation of metabolites are evaluated with the reagent of step a).

41-50. (canceled)
